(19) United States
(12) Patent Application Publication
(10) Pub. No.: US 2004/0116863 A1
(43) Pub. Date: Jun. 17, 2004

(54) Intra-venous catheter structure

(76) Inventors: Chang-Ming Yang, Miaoli (TW); Chun-Mei Chou, Miaoli (TW)

Correspondence Address: TROXELL LAW OFFICE PLLC, SUITE 1404, 5205 LEESBURG PIKE, FALLS CHURCH, VA, 22041, US

(21) Appl. No.: 10/317,054

(22) Filed: Dec. 12, 2002

(51) Int. Cl. A61M 5/178

(52) U.S. Cl. 604/164.01; 604/187

(57) ABSTRACT

An intra-venous catheter structure includes a syringe, a catheter, and a cap. The syringe has one or more buckle set at a side edge thereof. The cap has an opening set at a front end thereof and plural hooks set on a front end of the opening, plural sliding plates extendedly set on an outer edge of a back end of the cap corresponding to a position and a number of the one or more buckle of the syringe and a sliding trough formed on each of the plural of sliding plates, and a fastening portion formed on each the sliding trough at a portion approaching to a terminal end thereof. Furthermore, the catheter is sleeved on the socket of the syringe through the sleeve, the needle of the syringe is placed into the hose of the catheter, and through one or more buckle of the syringe which is respectively placed in each sliding trough, the cap is sliding on the syringe.

### BACKGROUND OF THE INVENTION

1. Field of the Invention

The present invention relates to an intra-venous catheter structure, and more particular to an intra-venous catheter structure for avoiding the operation risk of the medical personnel.

2. Description of the Prior Art

Generally, the conventional intra-venous catheter structure comprises a syringe, a catheter, and a cap, wherein the catheter is sleeved on the syringe, and the cap is covered around a front end of the syringe and also an outer edge of the catheter. When this conventional intra-venous catheter structure is under used, the cap will be taken off firstly, and the syringe with the catheter is injected into an injecting portion of a patient by a needle on the syringe. Then, a hose and a sleeve of the catheter will be pressed for pulling out the syringe and leaving the catheter at the injecting portion of the patient, so as to separate the catheter from the syringe. Finally, a conduit will be passed though the sleeve of the catheter for proceeding the injection. But, when the syringe and the catheter of this intra-venous catheter structure are separated, the blood of the patient will gush from the catheter. Thus, when the medical personnel draws out the syringe and injects the conduit into the sleeve of the catheter, the changing speed must be fast to avoid the blood gushing too much. However, because the speed of exchanging the syringe and the conduit is needed to be fast, the medical personnel is easy to be hurt by the drawn needle of the syringe which causes the risk.

Thus it can bee seen, the prior art described above still has some defects, is not a good design, however, and is urgently to be improved.

Because of the technical defects of the conventional intra-venous catheter structure described above, the applicant keeps on carving unflaggingly to develop an intra-venous catheter structure of the present invention through wholehearted experience and research.

### SUMMARY OF THE INVENTION

It is an object of the present invention to provide an intra-venous catheter structure for avoiding the operation risk of the medical personnel.

It is another object of the prevent invention to provide an intra-venous catheter structure for avoiding the backflow of the blood from the catheter when the syringe and the catheter are separated.

The intra-venous catheter structure for achieving the objects described above includes:

a syringe which has one or more buckle set at a side edge thereof and a blocking element plugged into a back end thereof;

a catheter which has a sleeve set at a front end thereof and a hose extended from a front end of the sleeve, and a resistant portion set around an outer edge of a back end of the sleeve;

a cap which has a opening at a front end thereof and plural hooks set on a front end of the opening, plural sliding plates extendedly set on an outer edge of a back end of the cap corresponding to a position and a number of one or more buckle of the syringe and a sliding trough formed on each of plural sliding plates, and a fastening portion formed on each sliding trough at a portion approaching to a terminal end thereof; and

one or more buckle of the syringe which is respectively placed in each sliding trough for sliding the cap on the syringe through the sliding trough.

### BRIEF DESCRIPTION OF THE DRAWINGS

The drawings disclose an illustrative embodiment of the present invention which serves to exemplify the various advantages and objects hereof, and are as follows:

FIG. 1 shows a three-dimensional perspective view of the intra-venous catheter structure according to the present invention;

FIG. 2 shows a three-dimensional appearance view of the intra-venous catheter structure according to the present invention;

FIG. 3 shows a schematic view of the three-dimensional motions of the intra-venous catheter structure according to the present invention;

FIG. 4 shows a side cross-sectional view of the three-dimensional actions of the intra-venous catheter structure according to the present invention;

FIG. 5 shows a side schematic view (I) of the motions of the intra-venous catheter structure according to the present invention;

FIG. 6 shows a side schematic view (II) of the motions of the intra-venous catheter structure according to the present invention;

FIG. 7 shows a side schematic view (III) of the motions of the intra-venous catheter structure according to the present invention;

FIG. 8 shows a side schematic view (IV) of the motions of the intra-venous catheter structure according to the present invention;

FIG. 9 shows a side schematic view (V) of the motions of the intra-venous catheter structure according to the present invention;

FIG. 10 shows a three-dimensional appearance view of the intra-venous catheter structure in a first preferred embodiment according to the present invention;

FIG. 11 shows a three-dimensional perspective view of the intra-venous catheter structure in a second preferred embodiment according to the present invention;

FIG. 12 shows a three-dimensional perspective view of the intra-venous catheter structure in a third preferred embodiment according to the present invention;

FIG. 13 shows a three-dimensional perspective view of the intra-venous catheter structure in a fourth preferred embodiment according to the present invention;

FIG. 14 shows a three-dimensional appearance view of the intra-venous catheter structure in a fourth preferred embodiment according to the present invention;

FIG. 15 shows a three-dimensional perspective view of the intra-venous catheter structure in a fifth preferred embodiment according to the present invention;

FIG. 16 shows a three-dimensional appearance view of the intra-venous catheter structure in a fifth preferred embodiment according to the present invention;

FIG. 17 shows a three-dimensional perspective view of the intra-venous catheter structure in a sixth preferred embodiment according to the present invention;

FIG. 18 shows a side schematic view (I) of the motions of the intra-venous catheter structure in a sixth preferred embodiment according to the present invention;

FIG. 19 shows a side schematic view (II) of the motions of the intra-venous catheter structure in a sixth preferred embodiment according to the present invention;

FIG. 20 shows a three-dimensional perspective view of the intra-venous catheter structure in a seventh preferred embodiment according to the present invention;

FIG. 21 shows a three-dimensional appearance view of the intra-venous catheter structure in a seventh preferred embodiment according to the present invention;

FIG. 22 shows a three-dimensional perspective view of the intra-venous catheter structure in an eighth preferred embodiment according to the present invention;

FIG. 23 shows a three-dimensional perspective view of the intra-venous catheter structure in a ninth preferred embodiment according to the present invention;

FIG. 24 shows a three-dimensional perspective view of the intra-venous catheter structure in a tenth preferred embodiment according to the present invention;

FIG. 25 shows a three-dimensional perspective view of the intra-venous catheter structure in an eleventh preferred embodiment according to the present invention;

FIG. 26 shows a three-dimensional perspective view of another intra-venous catheter structure according to the present invention;

FIG. 27 shows a three-dimensional perspective view of another intra-venous catheter structure in a first preferred embodiment according to the present invention;

FIG. 28 shows a three-dimensional perspective view of another intra-venous catheter structure in a second preferred embodiment according to the present invention;

FIG. 29 shows a schematic view of the motions of another intra-venous catheter structure in a second preferred embodiment according to the present invention;

FIG. 30 shows a three-dimensional perspective view of another intra-venous catheter structure in a third preferred embodiment according to the present invention;

FIG. 31 shows a three-dimensional perspective view of another intra-venous catheter structure in a fourth preferred embodiment according to the present invention;

FIG. 32 shows a three-dimensional perspective view of another intra-venous catheter structure in a fifth preferred embodiment according to the present invention;

FIG. 33 shows a three-dimensional perspective view of another intra-venous catheter structure in a sixth preferred embodiment according to the present invention;

FIG. 34 shows a three-dimensional perspective view of another intra-venous catheter structure in a seventh preferred embodiment according to the present invention;

FIG. 35 shows a three-dimensional perspective view of another intra-venous catheter structure in an eighth preferred embodiment according to the present invention;

FIG. 36 shows a side cross-sectional view of the catheter in a first preferred embodiment according to the present invention;

FIG. 37 shows a side cross-sectional view of the catheter in a second preferred embodiment according to the present invention; and

FIG. 33 shows a side schematic view of the motions of the catheter in a second preferred embodiment according to the present invention.

### DETAILED DESCRIPTION OF THE PREFERRED EMBODIMENT

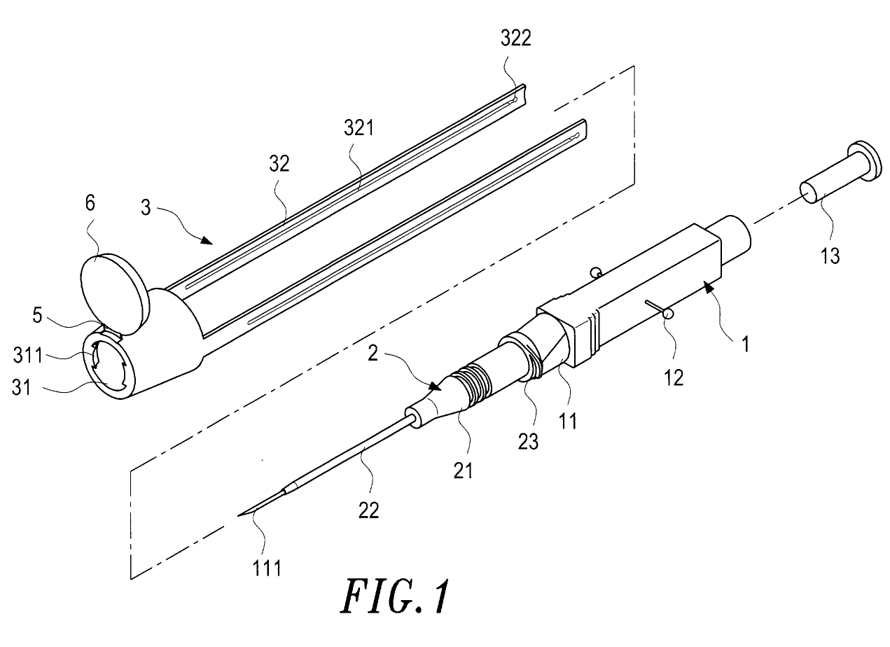

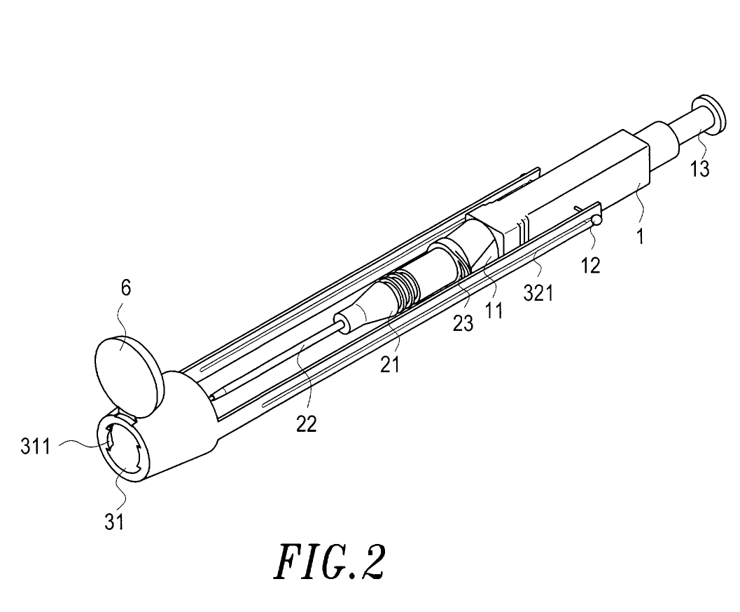

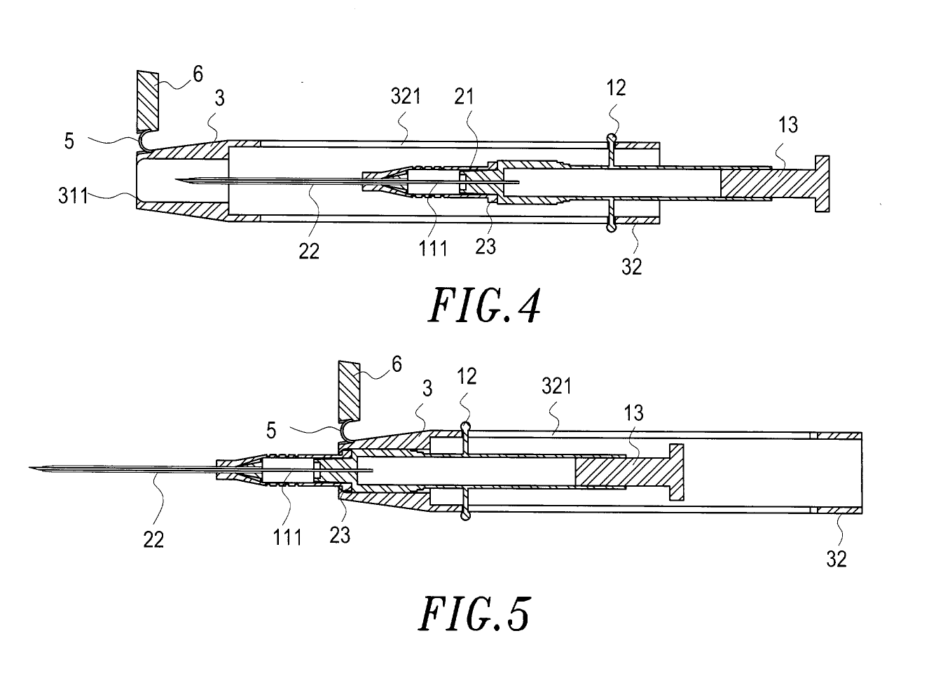

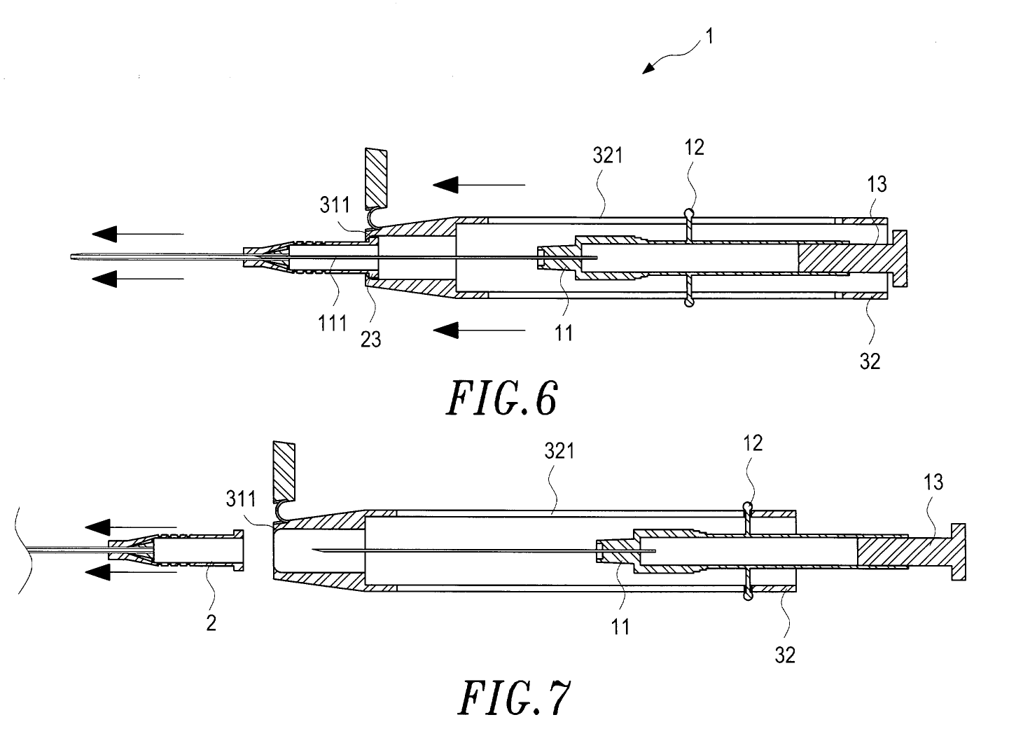

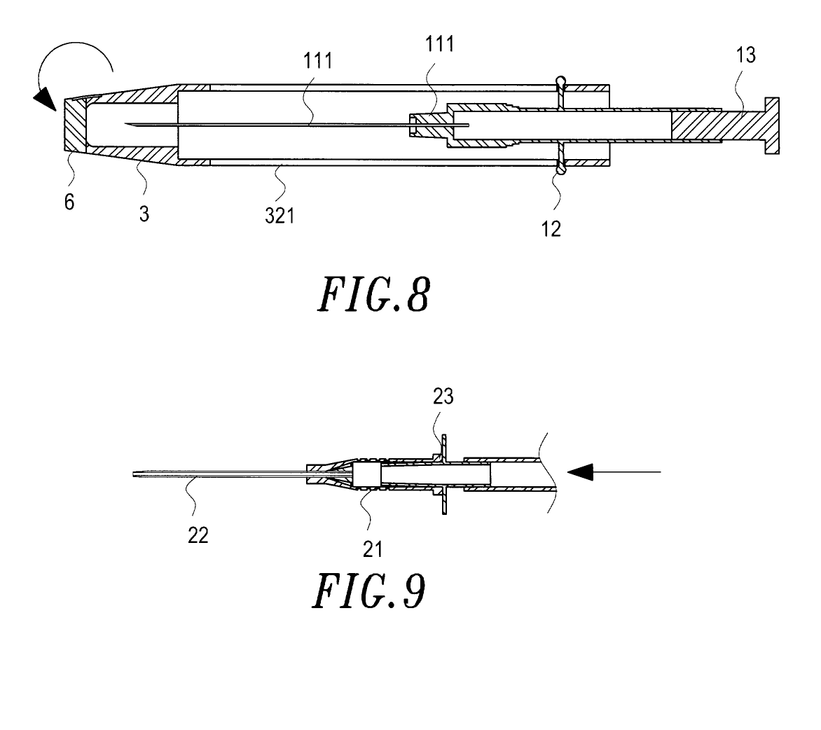

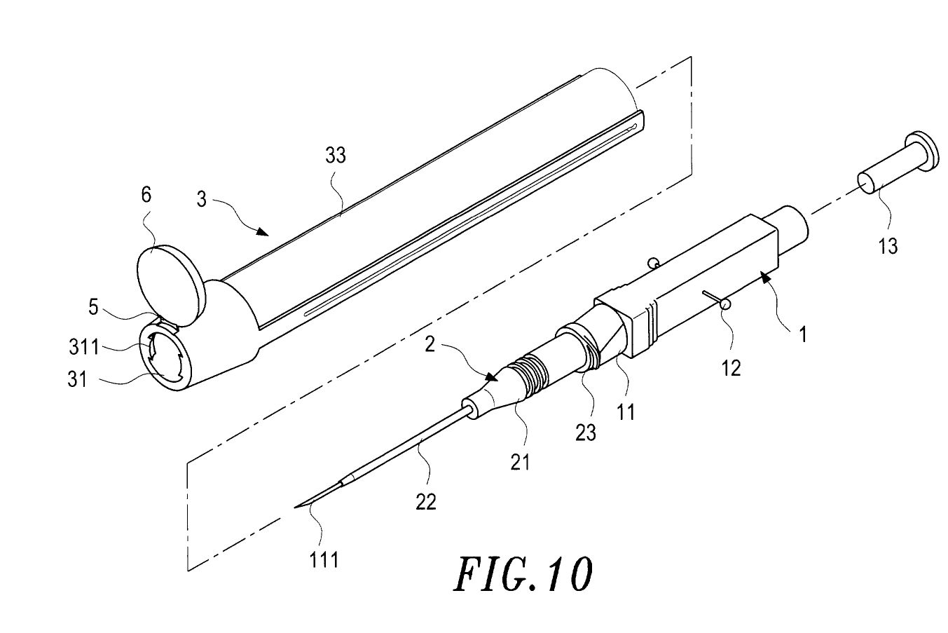

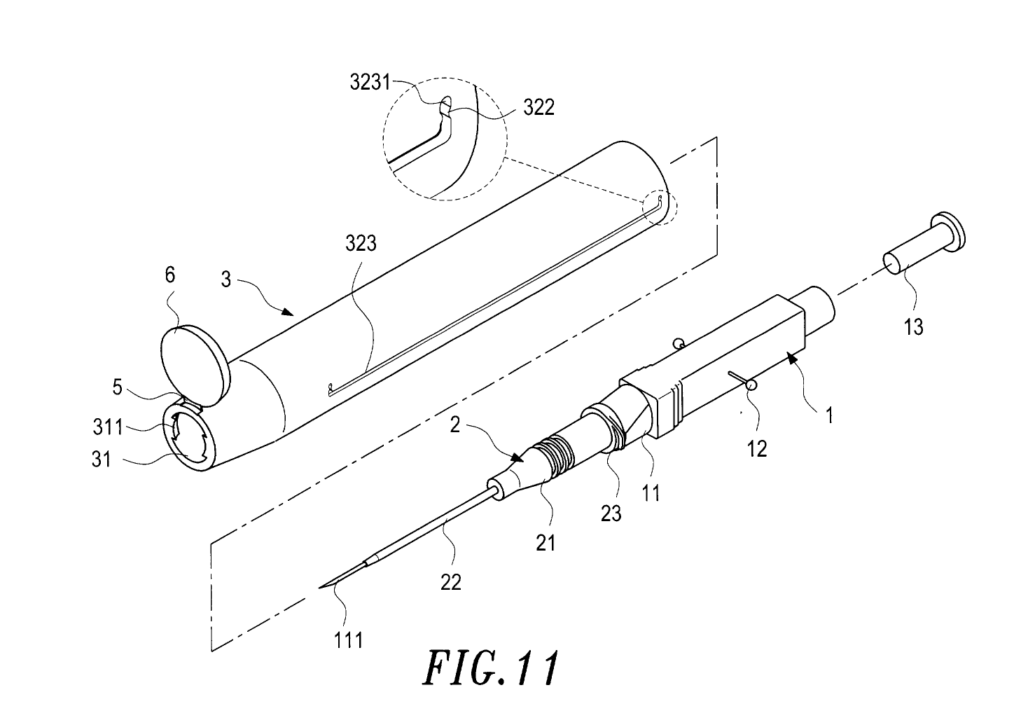

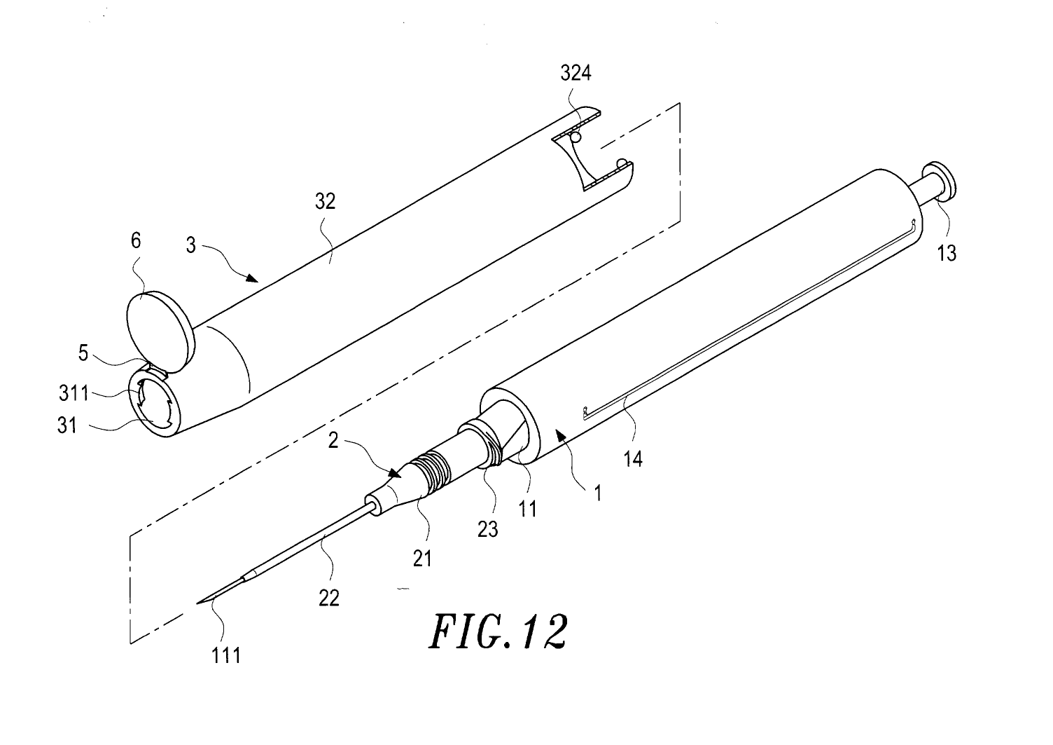

Please refer to FIGS. 1, 2, and 4 which illustrate the intra-venous catheter structure according to the present invention. The intra-venous catheter structure includes:

a syringe 1, wherein the syringe 1 has a socket 11 set at a front end thereof and a needle 111 set on the socket 11, one or more buckle 12 set at a side edge of the syringe 1, and a blocking element 13 plugged into a back end of the syringe 1;

a catheter 2, wherein the catheter 2 has a sleeve 21 set at a front end thereof and a hose 22 extended from a front end of the sleeve 21 and a resistant portion 23 set around an outer edge of a back end of the sleeve 21, and the catheter 2 is sleeved on the socket 11 of the syringe 1 through the sleeve 21 and the needle 111 of the syringe 1 is placed into the hose 22 of the catheter 2;

a cap 3, wherein the cap 3 has a opening 31 set at a front end thereof and plural hooks 311 set on a front end of the opening 31, plural sliding plates 32 extendedly set on an outer edge of a back end of the cap 3 corresponding to a position and a number of the one or more buckle 12 of the syringe 1 and a sliding trough 321 formed on each of plural sliding plates 32, and a fastening portion 322 formed on each of the sliding trough 321 at a portion approaching to a terminal end thereof, and the front end of the cap 3 has a connecting element 5 for connecting to a cover plate 6.

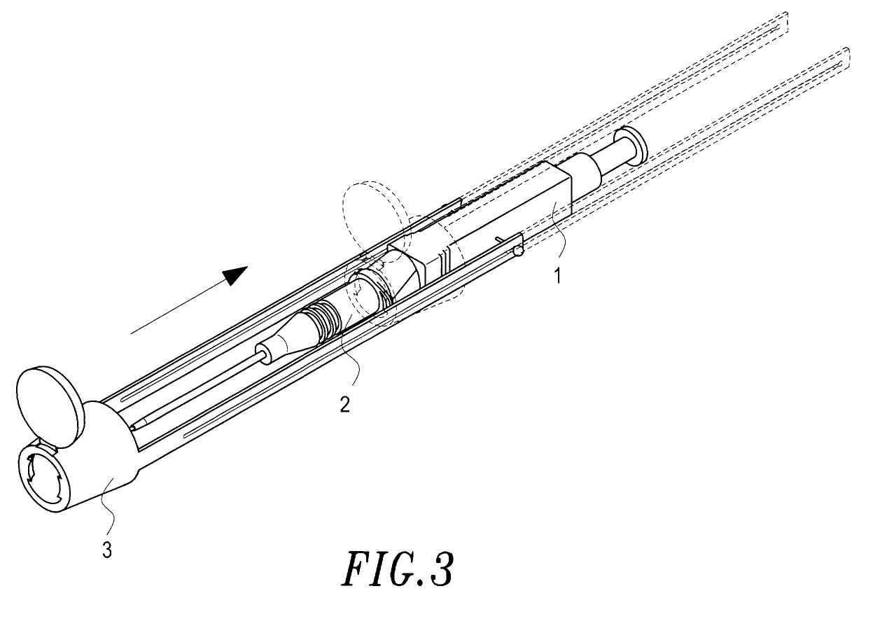

Please refer to FIGS. 3 and 5˜12. In the intra-venous catheter structure according to the present invention, each of one or more buckle 12 on the syringe 1 is placed into each sliding trough 321 of the cap 3. When the intra-venous catheter structure according to the present invention is under used, the covering plate 6 of the cap 3 is opened firstly, and then the syringe 1 will be slid in the sliding trough 321 of the cap 3 by means of one or more buckle 12. Because the terminal end of the sliding trough 321 has the fastening portion 322, one or more buckle 12 must be slightly pushed for passing through the fastening portion 322. The syringe 1 is slid in the sliding trough 321 of the cap 3 by means of one or more buckle 12, thus the cap 3 will be moved back to the back end of the syringe 1 and the needle 111 and the catheter 2 will be punched out from the opening 31 of the cap 3. Moreover, when the cap 3 is moved back to the back end of the syringe 1, plural hooks 311 set on the opening 31 of the front end of the cap 3 will hook the front edge of the resistant portion 23 on the sleeve 21. At this time, the needle 111 with the hose 22 of the catheter 2 is injected into the injecting portion of the patient, the sleeve 21 and the hose 22 of the catheter 2 are pressed for drawing out the syringe 1 and leaving the catheter 2 in the injecting portion, and then a conduit is passed though the sleeve 21 of the catheter 2 for proceeding the injection. Because plural hooks 311 set on the cap 3 hook the outer edge of the front end of the resistant portion 23 on the sleeve 21, when the syringe 1 and the catheter 2 are separated, the cap 3 will slide forward for covering the whole needle 111 inside the cap 3. At this moment, the cap 3 can be separated from the catheter 2 by a relatively greater strength and one or more buckle 12 of the syringe 1 is fixed by the fastening portion 322 set on the back end of the sliding trough 321 so as to maintain the needle 111 to be covered by the cap 3. Through this structure, the medical personnel will not be hurt by the needle 111 when using the intra-venous catheter, so as to avoid the operation risk. Moreover, the intra-venous structure according to the present invention can also set an outer circling sleeve 33 on an inner edge of each of plural sliding plates 32 of the cap 3 for being sleeved on the outer edge of the needle 111. Furthermore, the sliding trough 321 set at the back end of the cap 3 can be substituted by a U-shaped sliding trough 323, and, through rotating the cap 3, one or more buckle 12 can be fastened into a containing portion 3231 set at a front end or a back end of the U-shaped sliding trough 323. Also, each containing portion 3231 sets the fastening portion 322 for fastening one or more buckle 12 so as to fix the cap 3 at the front end or the back end of the syringe 1. In addition, the back end of each of plural sliding plates 32 which is set on the cap 3 can also set plural fasteners 324 and the side edge of the syringe 1 can set plural U-shaped fillisters 14 corresponding to the number and positions of plural fasteners 324.

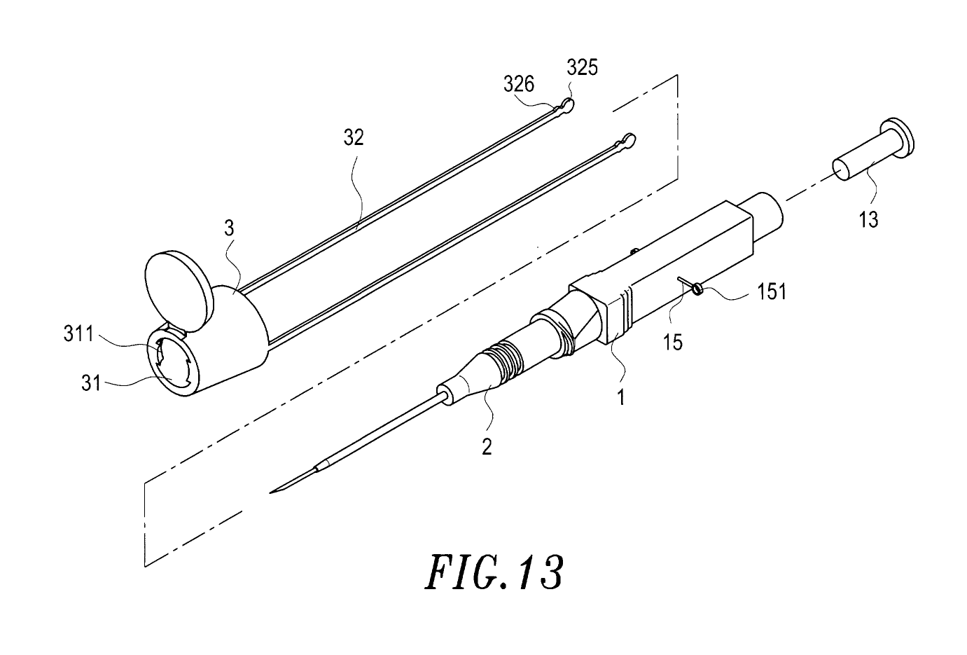

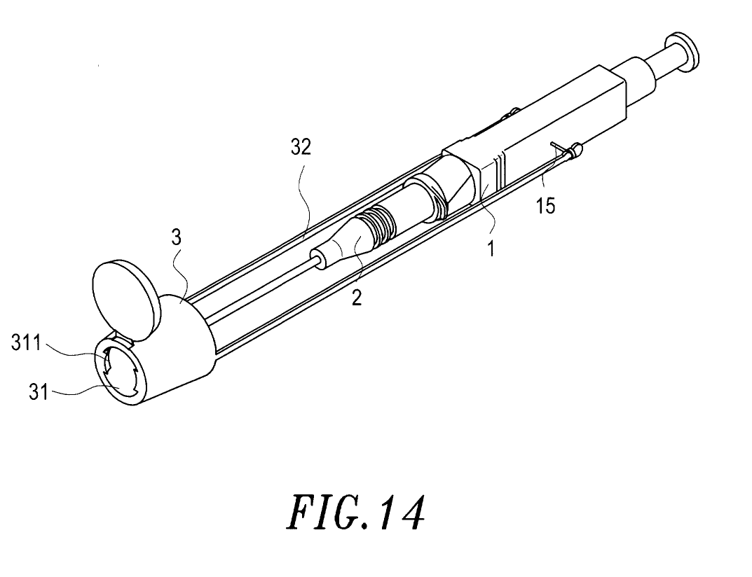

Please refer to FIGS. 13 and 14 which illustrate the intra-venous catheter structure according to the present invention. In the present invention, one or more buckle 12 of the syringe 1 can be substituted by a guiding structure 15 with a hole 151 for being passed through appropriately by plural sliding plates 32 which is set on the back end of the cap 3, and each of plural sliding plates 32 can set a retaining portion 325 at a terminal end thereof, wherein the retaining portion has a protrusion 326 at a front end thereof. Through setting the retaining portion 325, the cap 3 and the syringe 1 are uneasy to be separated. Furthermore, the purpose for setting the protrusion 326 is to maintain the needle 111 to be covered by the cap 3.

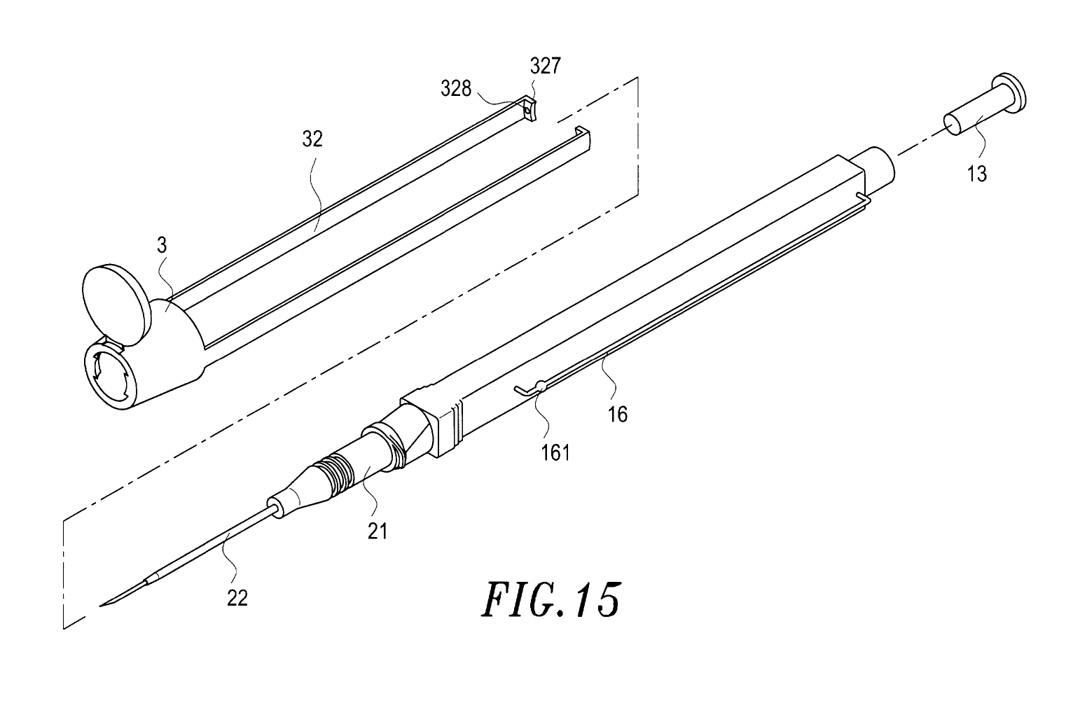

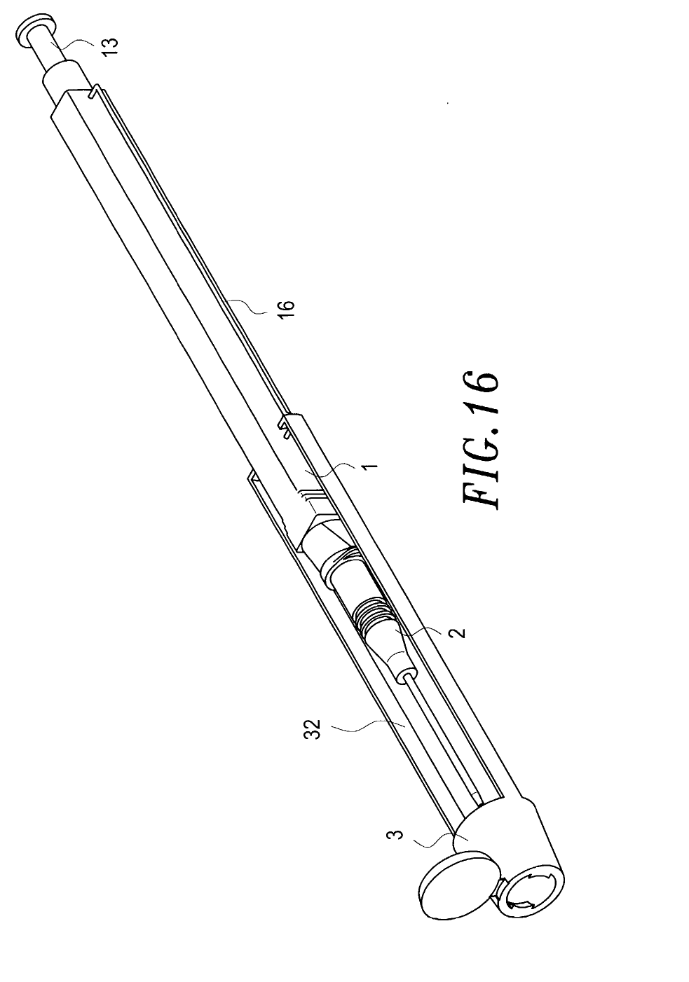

Please refer to FIGS. 15 and 16 which illustrate the intra-venous catheter structure according to the present invention. In the present invention, the syringe 1 can lack one or more buckle 12 but extendedly set plural guiding rails 16 on sides of the syringe 1, and each of plural guiding rails 16 can set a protruding circle 161 at a front end thereof. Additionally, each of plural sliding plates 32 which is set at the back end of the cap 3 has a protruding block 327 with a guiding hole 328 for being passed through by each the plural guiding rails 16.

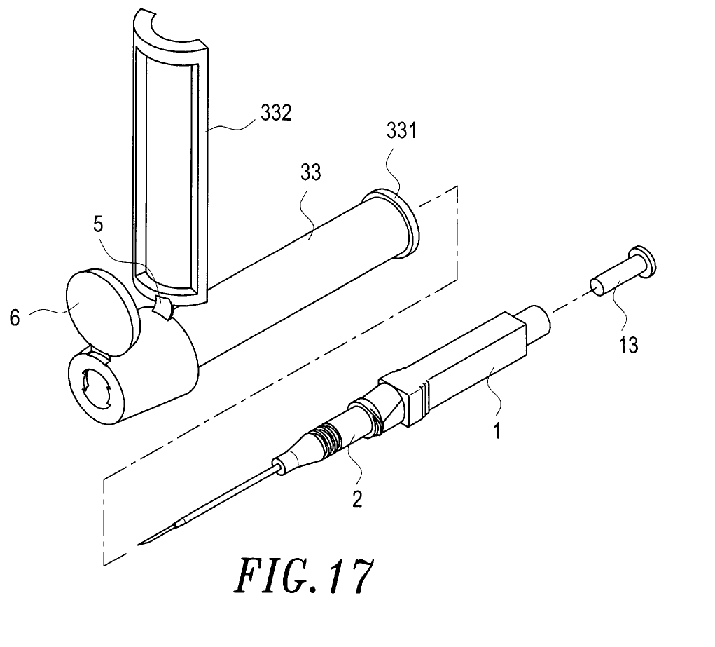

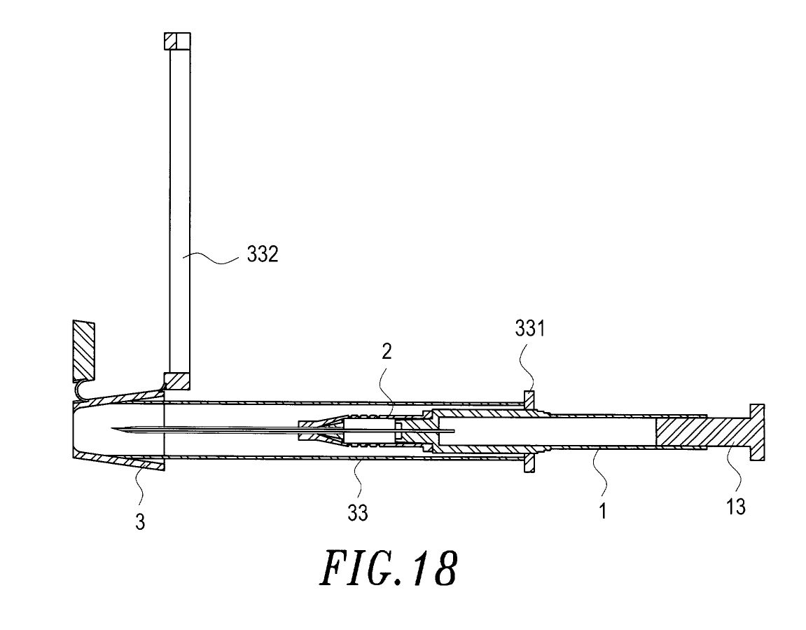

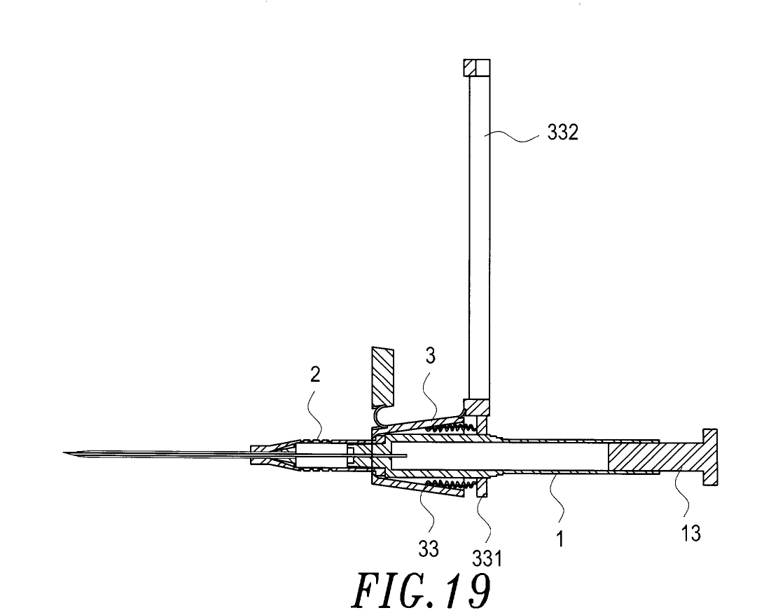

Please refer to FIGS. 17-19 which illustrate the intra-venous catheter structure according to the present invention. In the present invention, the syringe 1 can lack one or more buckle 12 but set an outer circling sleeve 33 made of a thin soft material at the back end of the cap 3 for connecting to a sleeving portion 331 which is sleeved on an outer edge of the syringe 1. The outer edge of the outer circling sleeve 33 is covered by a blocking structure 332 which is connected to the back end of the cap 3 through a connecting element 5 at a front end thereof, and the blocking structure 332 is rejected against the sleeving portion 331 of the syringe 1 by a back end thereof. Through utilizing the connecting element 5 as an axle to push the blocking structure 332 aside and then moving the cap 3 back, the outer circling sleeve 33 made of the soft material will shrink into the cap 3. When separating the catheter 2 and the cap 3, it only needs to push back the blocking structure 332 to reject between the cap 3 and the sleeving portion 331 for covering the needle 111 inside the cap 3.

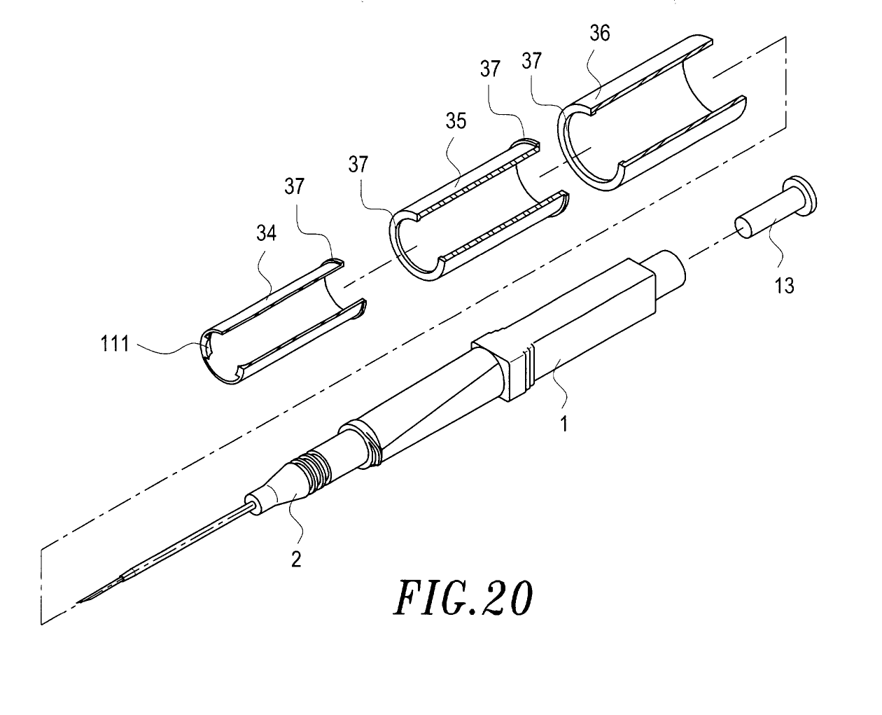

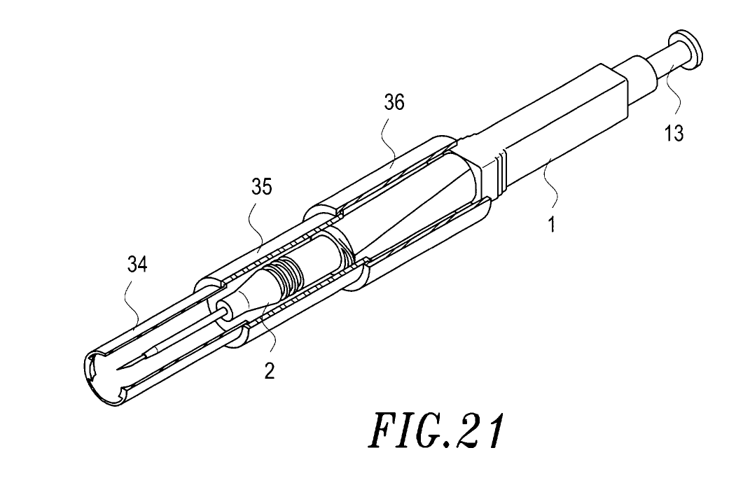

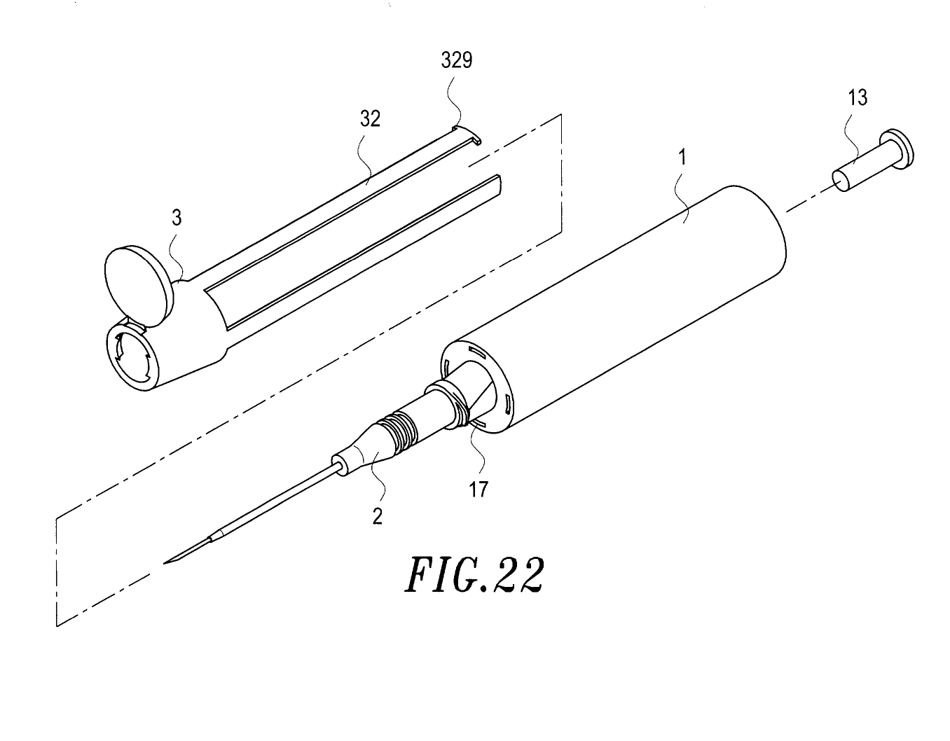

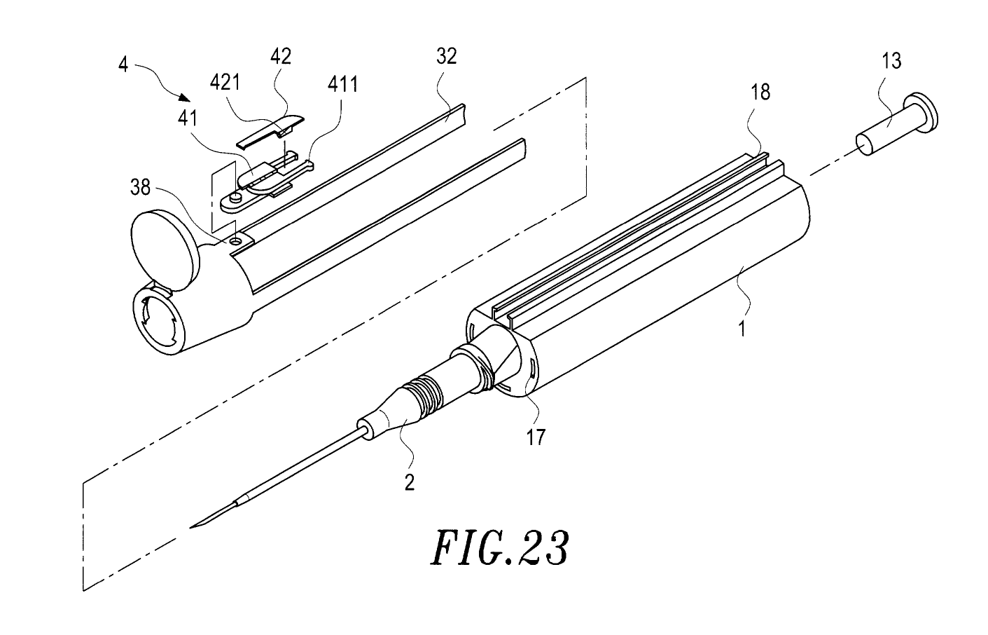

Please refer to FIGS. 20 and 21 which illustrate the intra-venous catheter structure according to the present invention. In the present invention, the syringe 1 can lack one or more buckle 12 but set an inner barrel sleeve 34, one or more intermediate barrel sleeve 35, and an outer barrel sleeve 36 instead. One or more intermediate barrel sleeve 35 is externally sleeved around the inner barrel sleeve 34, and the outer barrel sleeve 36 is externally sleeved around one or more intermediate barrel sleeve 35. The outer barrel sleeve 36 is sleeved on the outer edge of the syringe 1 through a back end thereof, the inner and one or more intermediate barrel sleeves 34 and 35 are both set a cycling protruding portion 37 at an outer edge of a back end thereof, and one or more intermediate barrel sleeve 35 and the outer barrel sleeve 36 both are set a cycling protruding portion 37 at an inner edge of a front end thereof. Furthermore, the inner barrel sleeve 34 is set plural hooks 311 at an inner edge of a front end thereof Please refer to FIGS. 22 and 23 which illustrate the intra-venous catheter structure according to the present invention. In the present invention, the syringe 1 can lack one or more buckle 12 but set plural guiding troughs 17 inside the syringe 1 for being passed through by each of plural sliding plates 32 of the cap 3, and any one or each of plural sliding plates 32 is set a stopping block 329 at a back end thereof. Otherwise, the syringe 1 can set a sliding rail 18 on any outer side thereof, and the cap 3 can has a connecting element 38 set at a position corresponding to the sliding rail 18 for connecting to a transposing component 4, wherein the transposing component 4 comprises a main body 41 and a fixing plate 42. The main body 41 is extendedly set two rejecting blocks 411 at a back end thereof, and the fixing plate 42 is set a circling protruding portion 412 at a bottom end thereof.

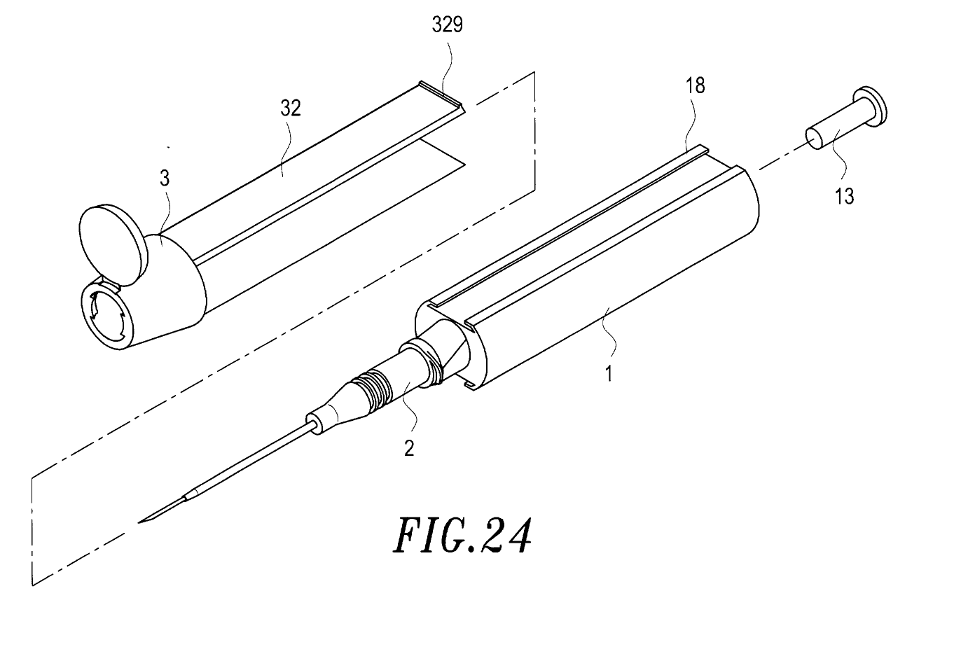

Please refer to FIG. 24 which illustrates the intra-venous catheter structure according to the present invention. In the present invention, the syringe 1 can set plural sliding rails 17 at the outer edge thereof for being passed through by plural sliding plates 32 set on the back end of the cap 3, and any one or each of plural sliding plates 32 is set a stopping block 327 at the back end thereof.

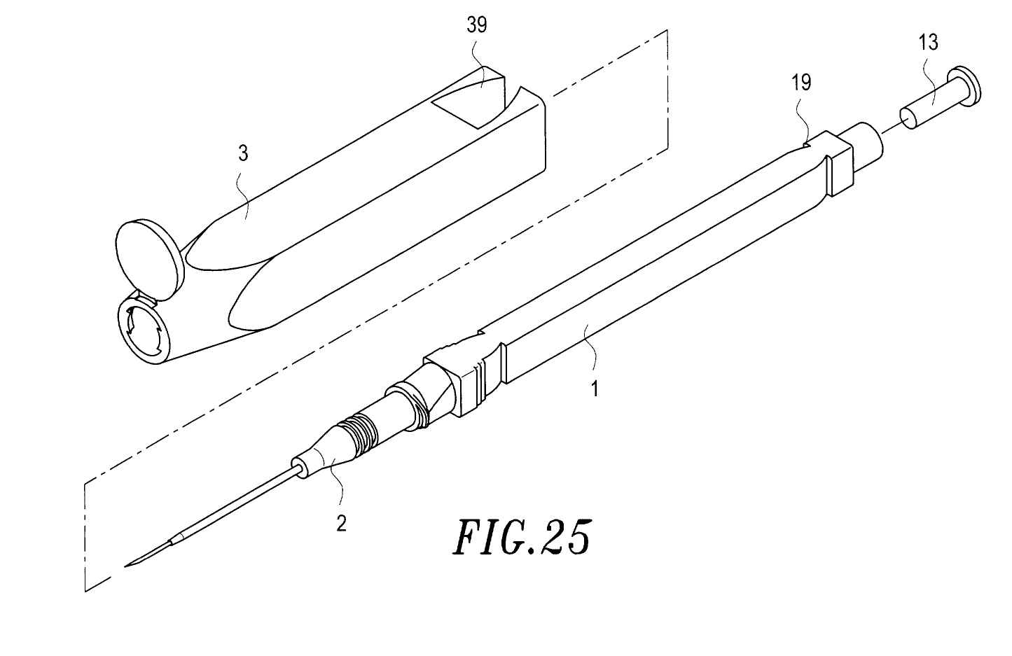

Please refer to FIG. 25 which illustrates the intra-venous catheter structure according to the present invention. In the present invention, the syringe 1 can lack one or more buckle 12 but concavely set plural placing portions 19 respectively on the outer edge of the front and the back ends of the syringe 1, and the cap 3 can set a buckling hook 39 for hooking inside plural placing portions 19 of the syringe 1 at the terminal end thereof and fixing the cap 3 at the front end or the back end of the syringe 1.

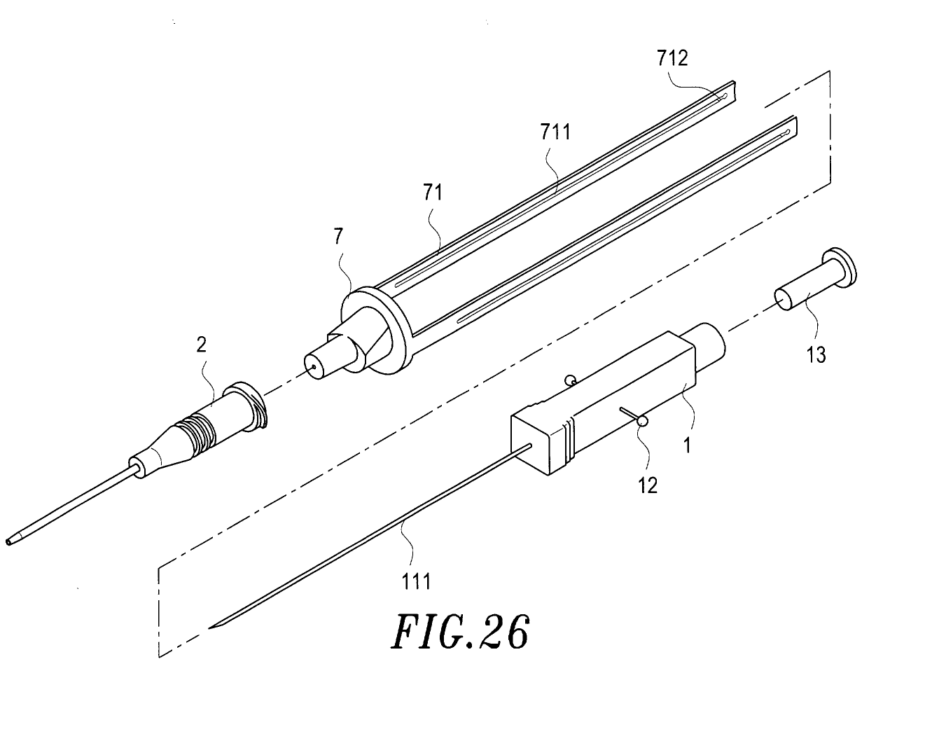

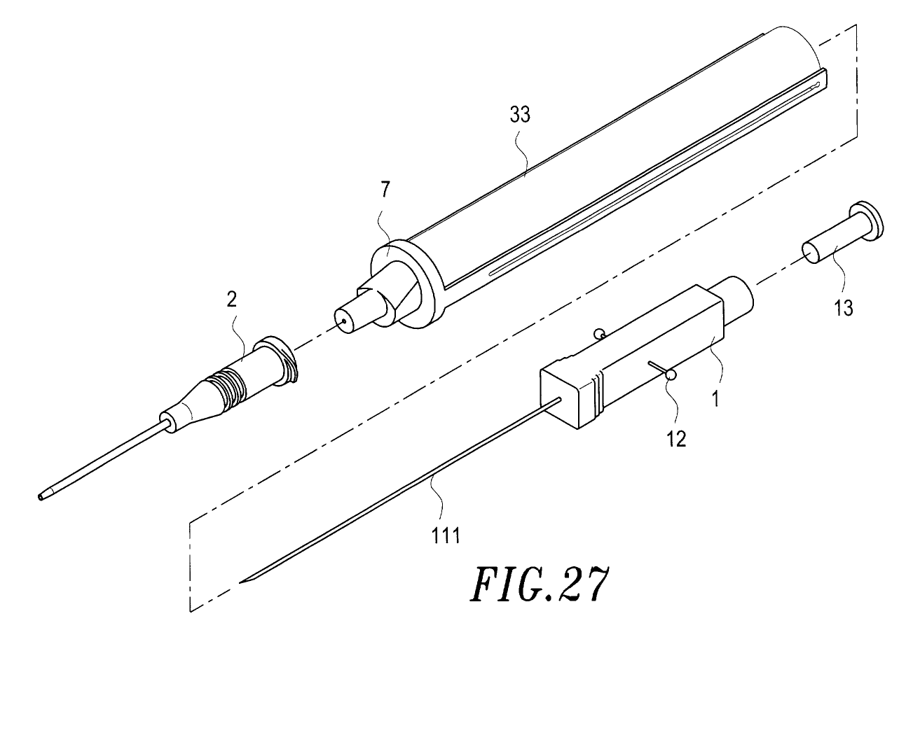

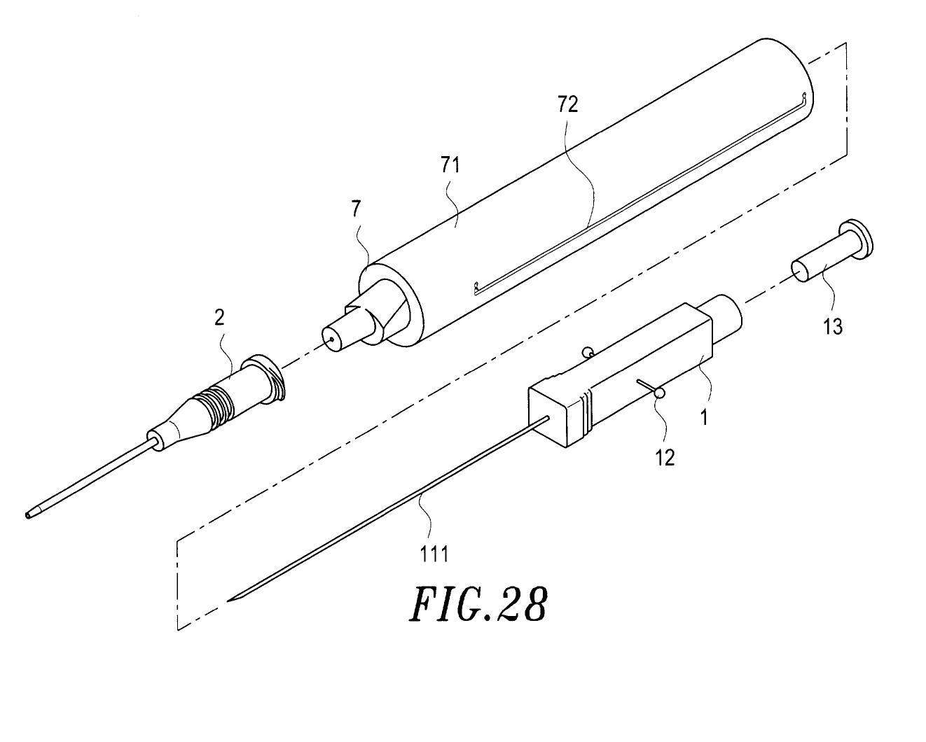

Please refer to FIGS. 26 and 27 which illustrate the intra-venous catheter structure according to the present invention. The intra-venous catheter structure includes:

a syringe 1, wherein the syringe 1 has a needle 111 set at a front end thereof, one or more buckle 12 set at a side edge of the syringe 1, and a blocking element 13 plugged into a back end of the syringe 1;

a catheter 2, wherein the catheter 2 has a sleeve 21 set at a front end thereof and a hose 22 extended from a front end of the sleeve 21, and a resistant portion 23 set around an outer edge of a back end of the sleeve 21;

a socket 7, wherein the socket 7 has plural sliding plates 71 extendedly set on an outer edge of a back end thereof corresponding to a position and a number of one or more buckle 12 of the syringe 1, a sliding trough 711 formed on each of plural sliding plates 71, and a fastening portion 712 formed on each sliding trough 713 at a portion approaching to a terminal end thereof; and

the socket 7 is sleeved on the front end of the syringe, the needle on the syringe is passed through the socket 7, the catheter is sleeved on the socket 7 through the sleeve, and one or more buckle of the syringe is respectively placed into each sliding trough 71 of the socket 7 for sliding the socket 7 on the syringe through the sliding trough 711; and that, the intra-venous catheter structure according to the present invention can also set an outer circling sleeve 33 on an inner edge of each of plural sliding plates 711 of the socket 7 for being sleeved on the outer edge of the needle 111.

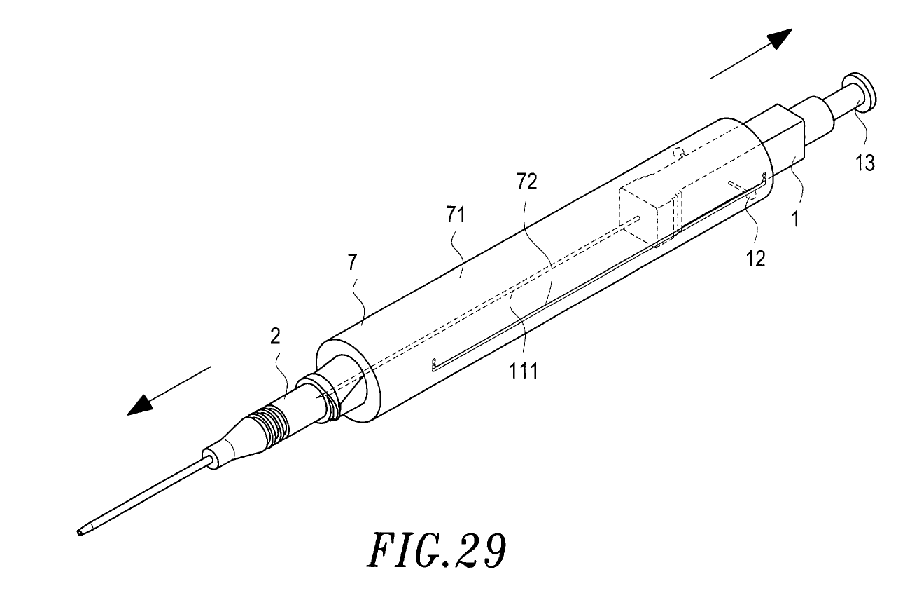

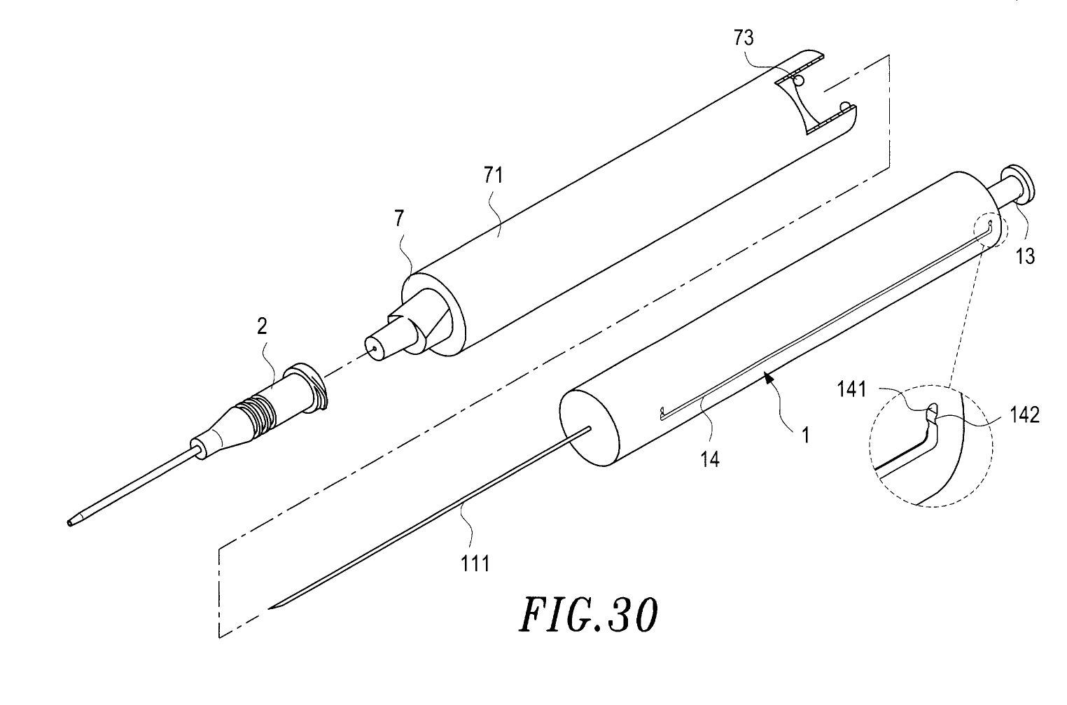

Please refer to FIGS. 28-30 which illustrate the intra-venous catheter structure according to the present invention. In the present invention, the sliding trough 711 set at each of plural sliding plates 71 which is extendedly set at the back end of the socket 7 can be substituted by a U-shaped sliding trough 72. In addition, the back end of each of plural sliding plates 71 which is set on the socket 7 can also set plural fasteners 73, and the side edge of the syringe 1 can set plural U-shaped fillisters 14 corresponding to the number and positions of plural fasteners 73. Through rotating the socket 7, plural fasteners 73 can be fastened into a containing portion 141 set at a front end or a back end of the U-shaped sliding trough 72, and, through the fastening portion 142 set inside the containing portion 141 fastening plural fasteners 73, the socket 7 can be fixed at the front end or the back end of the syringe 1.

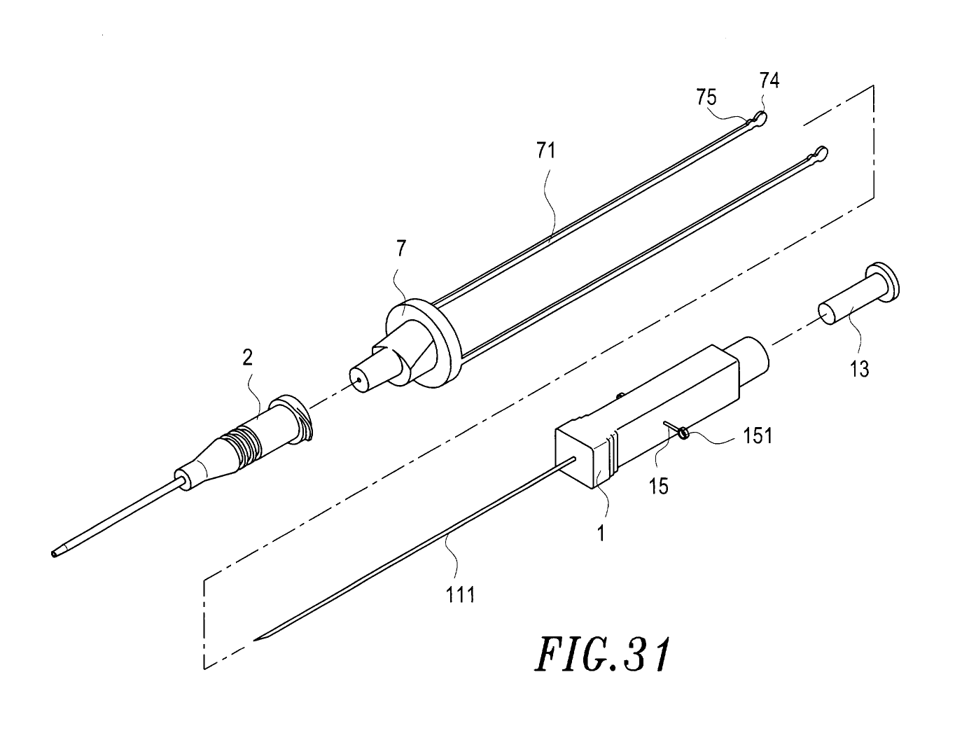

Please refer to FIG. 31 which illustrates the intra-venous catheter structure according to the present invention. In the present invention, one or more buckle 12 of the syringe 1 can be substituted by a guiding structure 15 with a hole 151 for being passed through appropriately by plural sliding plates 71 set on the back end of the socket 7, and each of plural sliding plates 71 is set a retaining portion 74 at a terminal end thereof, wherein the retaining portion 74 has a protrusion 75 at a front end thereof.

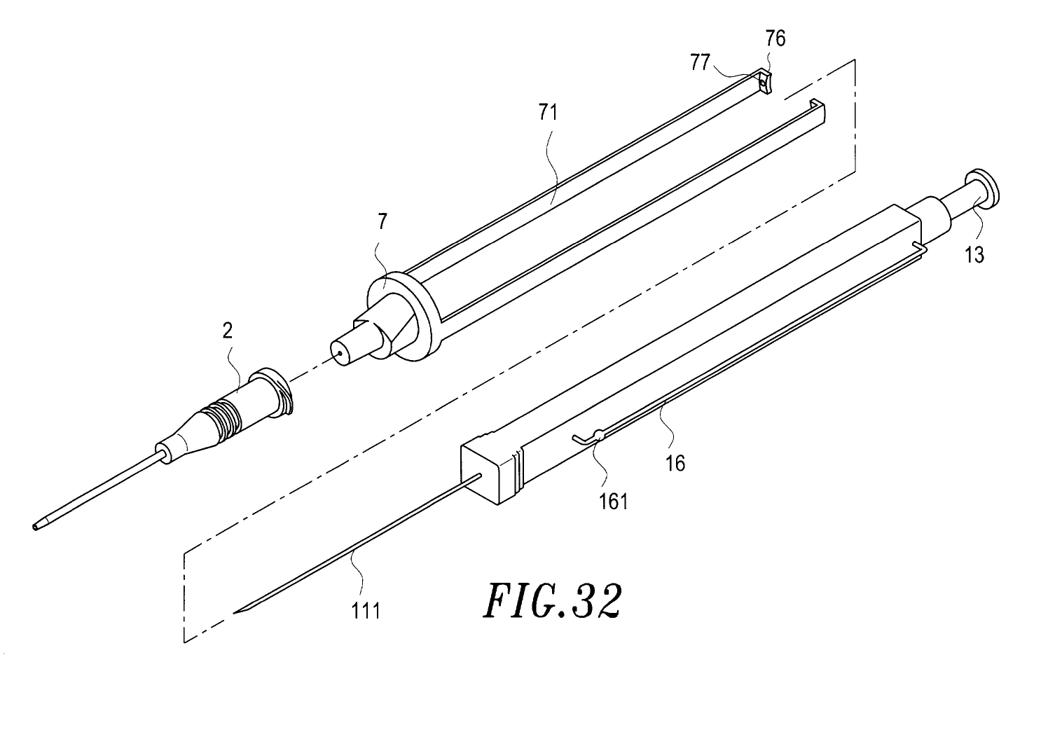

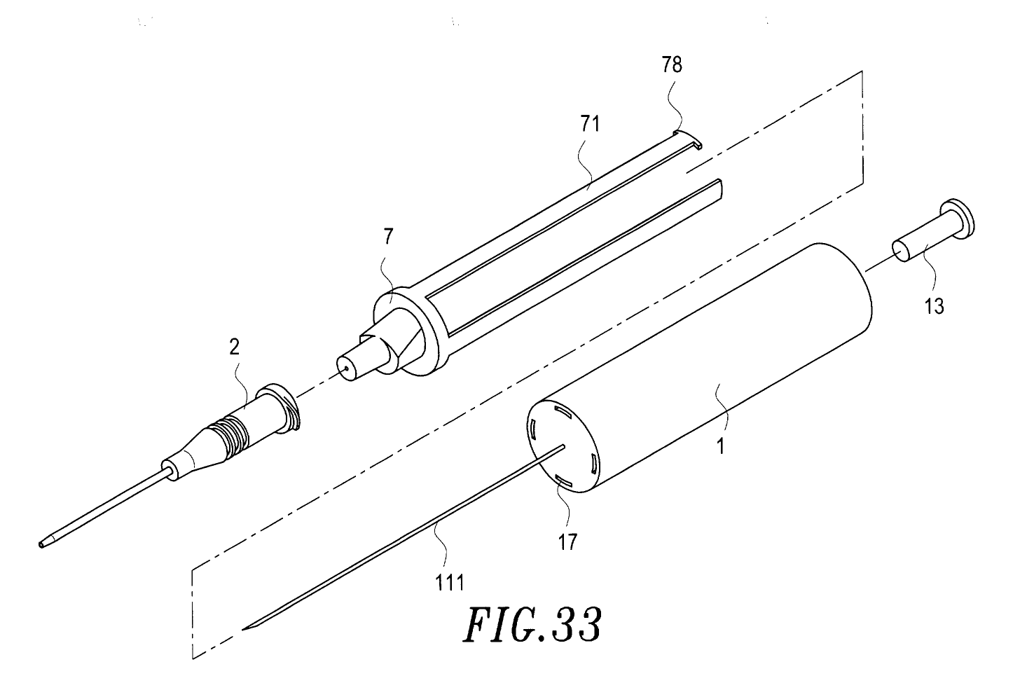

Please refer to FIG. 32 which illustrates the intra-venous catheter structure according to the present invention. In the present invention, the syringe 1 can lack one or more buckle 12 but extendedly set plural guiding rails 16 on sides of the syringe 1 and each of plural guiding rails 16 can set a protruding circle 161 at a front end thereof. Additionally, each of plural sliding plates 71 at the back end of the socket 7 can set a protruding block 76 with a guiding hole 77 for being passed through by each of plural guiding rails 16.

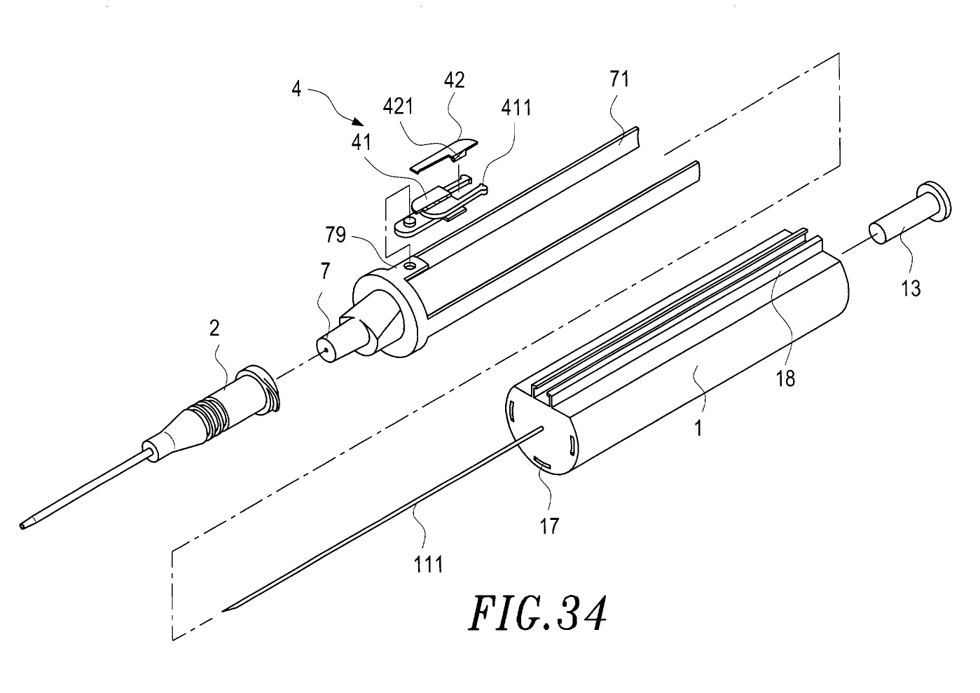

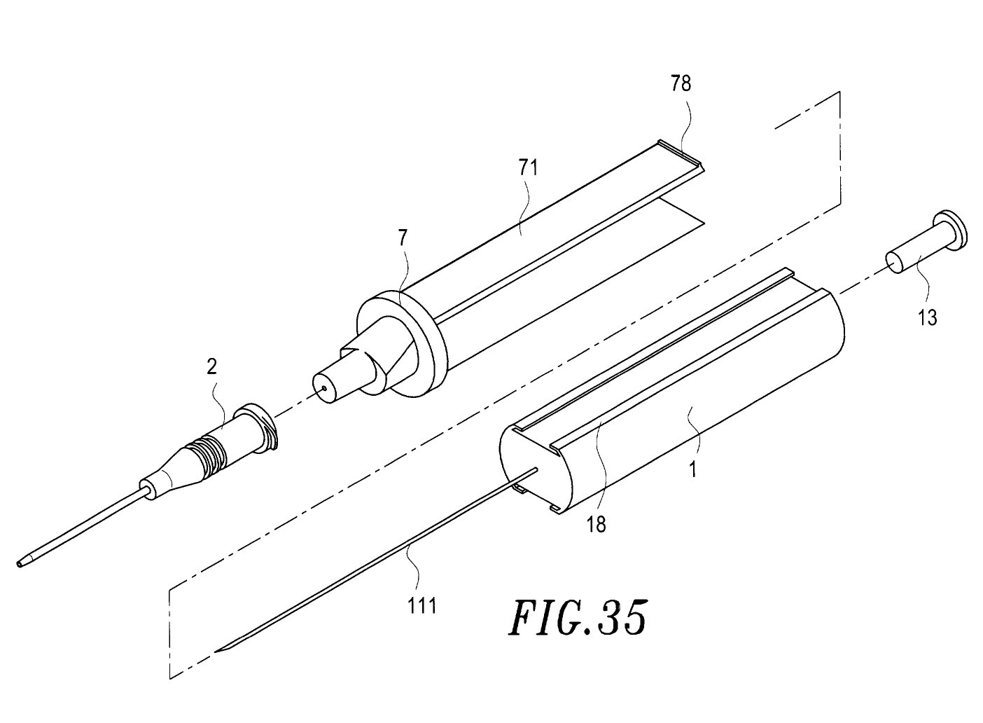

Please refer to FIGS. 33 and 34 which illustrate the intra-venous catheter structure according to the present invention. In the present invention, the syringe 1 can lack one or more buckle 12 but set plural guiding troughs 17 inside the syringe 1 for being passed through by plural sliding plates 71 of the socket 7, and any one or each of plural sliding plates 71 can set a stopping block 78 at a back end thereof. Otherwise, the syringe 1 can set a sliding rail 18 on any outer side thereof, and a connecting element can be set on a position of said socket 7 corresponding to the sliding rail 17 for connecting to a transposing component 4, wherein the transposing component 4 comprises a main body 41 and a fixing plate 42. The main body 41 is extendedly set two rejecting blocks 411 at a back end thereof, and the fixing plate 42 is set a circling protruding portion 412 at a bottom end thereof.

Please refer to FIG. 35 which illustrates the intra-venous catheter structure according to the present invention. In the present invention, the syringe 1 can set plural sliding rails 18 at the outer edge thereof for being passed through by plural sliding plates 71 which is set on the back end of the socket 7, and any one or each of plural of sliding plates 71 can set a stopping block 78 at the back end thereof.

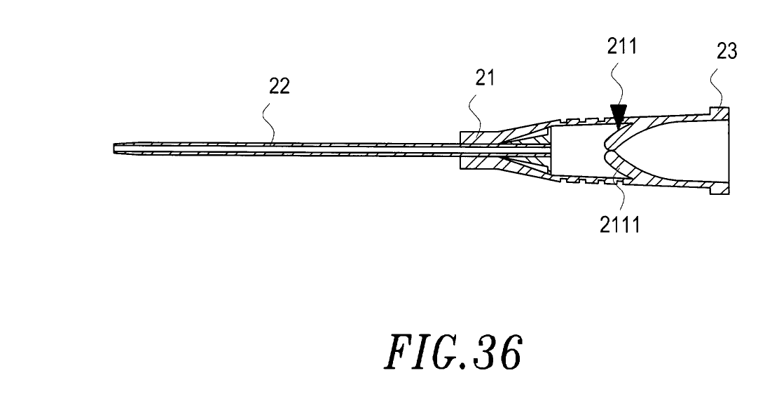

Please refer to FIG. 36 which illustrates the intra-venous catheter structure according to the present invention. In the present invention, a non-return structure 211 can be formed at an inner edge of the sleeve 21 of the catheter 2 by surrounding plural blades 2111. During the utilization of the intra-venous catheter structure, each of plural blades 2111 inside the catheter 2 will close to each other for avoiding a backflow of the blood from the catheter 2 when the syringe 1 and the catheter 2 are separated. Furthermore, when the conduit is passed through the sleeve 21, plural blades 2111 will be propped up by the conduit for benefiting the injection.

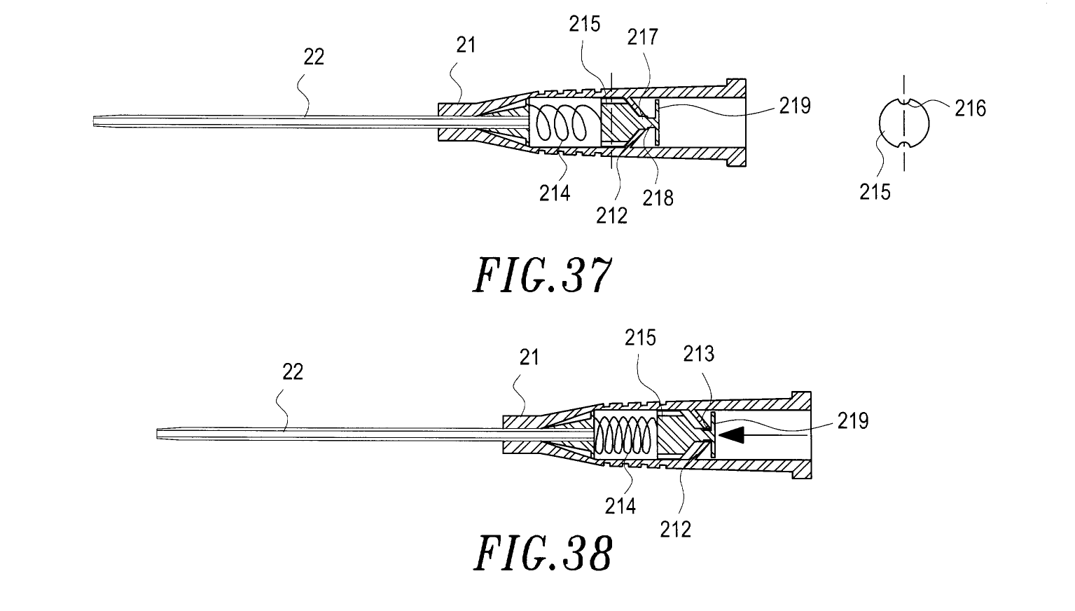

Please refer to FIGS. 37 and 38 which illustrate the intra-venous catheter structure according to the present invention. In the present invention, the sleeve 21 of the catheter 2 can internally set a block 212 with a passing hole 213, and an inner edge of a front end of the sleeve 21 is connected to a piston 215 made of an elastic material by a spring 214. The piston 215 is set plural diverting troughs 216 at a main body thereof and a back end of the piston 215 is placed in the passing hole 213 of the block 212 through a rejecting element 217, wherein the rejecting element 217 has a diverting portion 218 which has an external diameter smaller than that of the rejecting element 217 at a back end thereof, and the diverting portion 218 is extendedly set a rejecting block 219 at an outer edge thereof. When the intra-venous catheter is out of use, the needle 111 of the syringe 1 will being passed through the piston 215. Moreover, when the syringe 1 and the catheter 2 are separated, the spring 214 inside the catheter 2 will being moved back to push back the piston 215 so as to make the rejecting element 217 to reject against the passing hole 213 of the block 212 for avoiding the backflow of the blood from the catheter 2. Furthermore, when the conduit is passed through the sleeve 21, the conduit will therefore push the rejecting block 219 of the piston 215 so as to make the diverting portion 218 of the piston 215 to move into the passing hole 213 of the block 212. And, because the external diameter of the diverting portion 218 is smaller than that of the passing hole 213, the medicine can pass through the passing hole 213 and plural diverting troughs 216 of the piston 215 for proceeding the injection.

The intra-venous catheter structure according to the present invention, when being compared with the above-described cited reference and other prior arts, further includes the advantages as follows:

1. In the present invention, when the syringe and the catheter are separated, the cap will cover the needle so as to avoid the operation risk of the medical personnel.

2. According to the present invention, a non-return structure or a piston which is set inside the catheter can avoid the blood gushing from the catheter when the syringe and the catheter are separated.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. An intra-venous catheter structure, comprising:
a syringe, wherein a socket is set at a front end of said syringe and a needle is set on said socket, one or more buckle is set at a side edge of said syringe, and a blocking element is plugged into a back end of said syringe;
a catheter, wherein a sleeve is set at a front end of said catheter and a hose is extended from a front end of said sleeve, and a resistant portion is set around an outer edge of a back end of said sleeve;
a cap, wherein a opening is set at a front end of said cap and plural hooks are set at a front end of said opening, plural sliding plates are extendedly set on an outer edge of a back end of said cap corresponding to a position and a number of said one or more buckle of said syringe and a sliding trough is formed on each of said plural sliding plates, and a fastening portion is formed on each said sliding trough at a portion approaching to a terminal end thereof;
wherein said catheter is sleeved on said socket of said syringe through said sleeve, said needle of said syringe is placed into said hose of said catheter, said one or more buckle of said syringe is respectively placed in each said sliding trough for sliding said cap on said syringe through said sliding trough.

2. An intra-venous catheter structure according to claim 1, wherein said front end of said cap has a connecting element for connecting with a cover plate.

3. An intra-venous catheter structure according to claim 1, wherein said sliding trough which is set on said plural sliding plates extended from said back of said cap is capable of being substituted by a U-shaped sliding trough.

4. An intra-venous catheter structure according to claim 1, wherein said plural sliding plates set on said cap is capable of being set plural fasteners at said back end thereof and said syringe is capable of being set plural U-shaped fillisters corresponding to the number and positions of said plural fasteners at said side edge thereof.

5. An intra-venous catheter structure according to claim 1, wherein said one or more buckle of said syringe is capable of being substituted by a guiding structure with a hole for being passed through appropriately by said plural sliding plates which is set on said back end of said cap, and each of said plural sliding plates is set a retaining portion at a terminal end thereof and said retaining portion has a protrusion at a front end thereof.

6. An intra-venous catheter structure according to claim 1, wherein said syringe is capable of lacking said one or more buckle but extendedly setting plural guiding rails at sides of said syringe and each of said plural guiding rails is set a protruding circle at a front end thereof, and each of said plural sliding plates which is set at said back end of said cap has a protruding block with a guiding hole for being passed through by each of said plural guiding rails.

7. An intra-venous catheter structure according to claim 1, wherein said syringe is capable of lacking said one or more buckle but setting an outer circling sleeve made of a thin soft material at said back end of said cap for connecting to a sleeving portion which is sleeved on an outer edge of said syringe and an outer edge of said outer circling sleeve is covered by a blocking structure which is connected to said back end of said cap through a connecting element at a front end thereof, and said blocking structure is rejected against said sleeving portion of said syringe by a back end thereof.

8. An intra-venous catheter structure according to claim 1, wherein said syringe is capable of lacking said one or more buckle but setting an inner barrel sleeve, one or more intermediate barrel sleeve, and an outer barrel sleeve instead, said one or more intermediate barrel sleeve is externally sleeved around said inner barrel sleeve and said outer barrel sleeve is externally sleeved around said one or more intermediate barrel sleeve, wherein said outer barrel sleeve is sleeved on said outer edge of said syringe through a back end thereof, said inner and said one or more intermediate barrel sleeves are respectively set a cycling protruding portion at an outer edge of a back end thereof, said one or more intermediate and said outer barrel sleeves are respectively set a cycling protruding portion at an inner edge of a front end thereof, and said inner barrel sleeve is set plural hooks at an inner edge of a front end thereof.

9. An intra-venous catheter structure according to claim 1, wherein said syringe is capable of lacking said one or more buckle but setting plural guiding troughs inside said syringe for being passed through by said plural sliding plates of said cap, and any one or each of said plural sliding plates is set a stopping block at a back end thereof.

10. An intra-venous catheter structure according to claim 9, wherein said syringe is capable of setting a sliding rail on any outer side thereof, and a connecting element is set on a position of said cap corresponding to said sliding rail of said syringe for connecting to a transposing component, wherein said transposing component comprises a main body and a fixing plate, said main body is extendedly set two rejecting blocks at a back end thereof, and said fixing plate is set a circling protruding portion at a bottom end thereof.

11. An intra-venous catheter structure according to claim 1, wherein said syringe is capable of setting plural sliding rails at said outer edge thereof for being passed through by said plural sliding plates set on said back end of said cap, and any one or each of said plural sliding plates is set a stopping block at said back end thereof.

12. An intra-venous catheter structure according to claim 1, wherein said syringe is capable of lacking said one or more buckle but concavely setting plural placing portions respectively on said outer edge of said front and said back ends of said syringe, and said syringe is set a buckling hook for hooking inside said plural placing portions at said terminal end thereof.

13. An intra-venous catheter structure according to clam 1, wherein said sleeve of said catheter is formed a non-return structure at an inner edge thereof by surrounding plural blades.

14. An intra-venous catheter structure according to claim 1, wherein said sleeve of said catheter is internally set a block with a passing hole and an inner edge of a front end of said sleeve is connected to a piston made of an elastic material by a spring, said piston is set plural diverting troughs at a main body thereof and a back end of said piston is placed into said passing hole of said block through a rejecting element, said rejecting element is set a diverting portion which has an external diameter smaller than that of said rejecting element at a back end thereof, and said diverting portion is extendedly set a rejecting block at an outer edge thereof.

15. An intra-venous catheter structure, comprising:
a syringe, wherein a needle is set at a front end of said syringe, one or more buckle is set at a side edge of said syringe, and a blocking element is plugged into a back end of said syringe;
a catheter, wherein a sleeve is set at a front end of said catheter and a hose is extended from a front end of said sleeve, and a resistant portion is set around an outer edge of a back end of said sleeve;
a socket, wherein plural sliding plates are extendedly set at an outer edge of a back end of said socket corresponding to a position and a number of said one or more buckle of said syringe and a sliding trough is formed on each of said plural sliding plates, and a fastening portion is formed on each said sliding trough at a portion approaching to a terminal end thereof;
wherein said socket is sleeved on said front end of said syringe, said needle of said syringe is passed through said socket, said catheter is sleeved on said socket through said sleeve, and said one or more buckle of said syringe is respectively placed into each said sliding trough of said socket for sliding said socket on said syringe through said sliding trough.

16. An intra-venous catheter structure according to claim 15, wherein said sliding trough set on each of said plural sliding plates which is extendedly set on said back end of said socket is capable of being substituted by a U-shaped sliding trough.

17. An intra-venous catheter structure according to claim 15, wherein said plural sliding plates on said socket is capable of being set plural fasteners at said back end thereof, and said syringe is capable of being set plural U-shaped fillisters corresponding to the number and positions of said plural fasteners at said side edge thereof.

18. An intra-venous catheter structure according to claim 15, wherein said one or more buckle of said syringe is capable of being substituted by a guiding structure with a hole for being passed through appropriately by said plural sliding plates set on said back end of said socket, and each of said plural sliding plates is set a retaining portion at a terminal end thereof and said retaining portion has a protrusion at a front end thereof.

19. An intra-venous catheter structure according to claim 15, wherein said syringe is capable of lacking said one or more buckle but extendedly setting plural guiding rails on sides of said syringe and each of said plural guiding rails is set a protruding circle at a front end thereof, and each of said plural sliding plates which is set at said back end of said socket has a protruding block with a guiding hole for being passed through by each of said plural guiding rails.

20. An intra-venous catheter structure according to claim 15, wherein said syringe is capable of lacking said one or more buckle but setting plural guiding troughs inside said syringe for being passed through by said plural sliding plates of said socket, and any one or each of said plural sliding plates is set a stopping block at a back end thereof.

21. An intra-venous catheter structure according to claim 15, wherein said syringe is capable of setting a sliding rail on any outer side thereof, and a connecting element is set on a position of said socket corresponding to said sliding rail of said syringe for connecting to a transposing component, wherein said transposing component comprises a main body and a fixing plate, said main body is extendedly set two rejecting blocks at a back end thereof, and said fixing plate is set a circling protruding portion at a bottom end thereof.

22. An intra-venous catheter structure according to claim 15, wherein said syringe is capable of setting plural sliding rails at said outer edge thereof for being passed through by said plural sliding plates set on said back end of said socket, and any one or each of said plural sliding plates is set a stopping block at said back end thereof.

23. An intra-venous catheter structure according to claim 15, wherein said syringe is capable of lacking said one or more buckle but concavely setting plural placing portions respectively on said outer edge of said front and said back ends of said syringe, and said socket is set a buckling hook for hooking inside said plural placing portions at said terminal end thereof.

24. An intra-venous catheter structure according to clam 15, wherein said sleeve of said catheter is formed a non-return structure at an inner edge thereof by surrounding plural blades.

25. An intra-venous catheter structure according to claim 15, wherein said sleeve of said catheter is internally set a block with a passing hole and an inner edge of a front end of said sleeve is connected to a piston made of an elastic material by a spring, said piston is set plural diverting troughs at a main body thereof and a back end of said piston is placed into said passing hole of said block through a rejecting element, said rejecting element is set a diverting portion which has an external diameter smaller than that of said rejecting element at a back end thereof, and said diverting portion is extendedly set a rejecting block at an outer edge thereof.
